(19) United States
(12) Patent Application Publication
(10) Pub. No.: US 2025/0006316 A1
(43) Pub. Date: Jan. 2, 2025

(54) SYSTEMS AND METHODS FOR PROVIDING A DISTRIBUTED PLATFORM WITH SEQUENTIAL RANDOMIZED TRIALS

(71) Applicant: Optum, Inc., Minnetonka, MN (US)

(72) Inventors: Laura D. HAMILTON, Chicago, IL (US); Spencer Tyler HA, Revere, MA (US); Fazle Shahnawaz Muhibul KARIM, Chicago, IL (US)

(21) Appl. No.: 18/342,972

(22) Filed: Jun. 28, 2023

(51) Int. Cl. G16H 10/20; G16H 10/60; G16H 40/20

(52) U.S. Cl. CPC G16H 10/20; G16H 10/60; G16H 40/20

(57) ABSTRACT

Executing a sequential randomized trial includes: obtaining a plurality of treatment paths, each including an ordered list of a plurality of treatment stages, assigning each of a plurality of subjects to a respective treatment path, for each subject, causing a corresponding user device to output a respective treatment corresponding to a first treatment stage of the respective treatment path, collecting response data, and performing at least one sequential iteration. The sequential iteration includes: identifying, based on the response data, at least one subject for which the respective treatment was ineffective, shifting the at least one subject to a next treatment stage on the respective treatment path, causing the corresponding user device of the at least one subject to output a further respective treatment corresponding to a current treatment stage of the respective treatment path; and re-collecting the response data from the user device associated with the at least one subject.

### TECHNICAL FIELD

Various embodiments of this disclosure relate generally to a distributed messaging platform, and, more particularly, to systems and methods for providing sequential randomized trials.

### BACKGROUND

In many situations, testing is performed to evaluate performance between different options. For example, different treatments may be evaluated for effectiveness, different messages may be evaluated for engagement, etc. Conventionally, such options may be evaluated via a trial procedure such as A/B testing, multi-variate testing, or the like.

However, such conventional trial procedures may be sub-optimal, may not be adapted to a particular situation, or may be associated with undesirable effects. For example, in trials involving providing different options to people, the subjects of the trial are generally divided up into cohorts, and then are locked into their respective cohort for the extent of the trial. Thus, a subject may be locked into an undesirable option for the full extent of the trial despite early indications that the option is not effective for that subject. Moreover, conventional A/B or multi-variate testing generally takes a significant amount of time to acquire statistically relevant data, which may thus prolong the negative experience of some subjects. Further, conventional trials generally evaluate a treatment based on its mean effect, and thus may not account for individualized responses from different subjects.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for providing a distributed messaging platform, e.g., that is usable to provide a sequential randomized trial. Conventional trial procedures have been used to evaluate different options, such as evaluating performance of an existing option or control against a new option or change. However, such conventional trial procedures like A/B testing, multi-variate testing, or the like, generally lock subjects into one option for the extent of the trial, and also generally require a significant amount of time in order to gather statistically relevant data. This may result in subjects being locked into a less preferable option during the extent of the trial. Such conventional trial procedures may also not account for or provide analysis of time-varying contextual factors that may impact the effectiveness or preferability of different options over time.

Accordingly, improvements in technology relating to trials are needed. As will be discussed in more detail below, in various embodiments, a distributed messaging platform is used to provide a sequential randomized trial that, over the course of a trial, shifts subjects along a path of different options, e.g., until an effective option is applied.

According to certain aspects of the disclosure, methods and systems are disclosed for providing a distributed messaging platform, e.g., for providing sequential randomized trials.

In some aspects, the techniques described herein relate to a distributed messaging platform for providing a sequential randomized trial, including: at least one trial system that includes: at least one memory storing instructions; and at least one processor operatively connected to the at least one memory, and configured to execute the instructions to perform operations for executing the sequential randomized trial. The operations include: obtaining a plurality of treatment paths to be used in the sequential randomized trial, each treatment path including an ordered list of a plurality of treatment stages; assigning each subject of a plurality of subjects to a respective treatment path; for each subject, causing a corresponding user device to output a respective treatment corresponding to a first treatment stage of the respective treatment path; collecting, from the user devices, response data of the plurality of subjects; and performing at least one sequential iteration. The at least one sequential iteration includes: identifying, based on the response data, at least one subject for which the respective treatment was ineffective; shifting the at least one subject to a next treatment stage on the respective treatment path; causing the corresponding user device of the at least one subject to output a further respective treatment corresponding to a current treatment stage of the respective treatment path; and re-collecting the response data from the user device associated with the at least one subject.

In some aspects, the techniques described herein relate to a computer-implemented method of using one or more processors of a distributed messaging platform to provide a sequential randomized trial, including: obtaining a plurality of treatment paths to be used in the sequential randomized trial, each treatment path including an ordered list of a plurality of treatment stages; assigning each subject of a plurality of subjects to a respective treatment path; for each subject, causing a corresponding user device to output a respective treatment corresponding to a first treatment stage of the respective treatment path; collecting, from the user devices, response data of the plurality of subjects; and performing at least one sequential iteration. The at least one sequential iteration includes: identifying, based on the response data, at least one subject for which the respective treatment was ineffective; shifting the at least one subject to a next treatment stage on the respective treatment path; causing the corresponding user device of the at least one subject to output a further respective treatment corresponding to a current treatment stage of the respective treatment path; and re-collecting the response data from the user device associated with the at least one subject.

In some aspects, the techniques described herein relate to a non-transitory computer-readable medium including instructions for using a distributed messaging platform to provide a sequential randomized trial, the instructions executable by at least one processor to perform operations including: obtaining a plurality of treatment paths to be used in the sequential randomized trial, each treatment path including an ordered list of a plurality of treatment stages; assigning each subject of a plurality of subjects to a respective treatment path; for each subject, causing a corresponding user device to output a respective treatment corresponding to a first treatment stage of the respective treatment path; collecting, from the user devices, response data of the plurality of subjects; and performing at least one sequential iteration. The at least one sequential iteration includes: identifying, based on the response data, at least one subject for which the respective treatment was ineffective; shifting the at least one subject to a next treatment stage on the respective treatment path; causing the corresponding user device of the at least one subject to output a further respective treatment corresponding to a current treatment stage of the respective treatment path; and re-collecting the response data from the user device associated with the at least one subject.

It is to be understood that both the foregoing general description and the following detailed description include examples and are explanatory only and are not restrictive of the disclosed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 depicts an example environment for a distributed messaging platform usable to provide a sequential randomized trial, according to one or more embodiments.

FIG. 2 depicts an example embodiment of a method for implementing a sequential randomized trial, according to one or more embodiments.

FIG. 3 depicts another example embodiment of a method for implementing a sequential randomized trial, according to one or more embodiments

FIG. 4 depicts an example of a computing device, according to one or more embodiments.

### DETAILED DESCRIPTION OF EMBODIMENTS

Examples in this disclosure are made with reference to different options of subjects with regard to medical treatments, interventions, recommendations, etc. However, it should be understood that reference to any particular activity is provided in this disclosure only for convenience and as an example, and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

The term “provider” generally encompasses an entity or agent thereof involved in providing goods or services to a person, e.g., healthcare to a patient. The term “treatment” generally encompasses application of an option in a trial to a subject. In other words, a “treatment” need not be medical in nature, although medical treatments such as interventions, prescriptions, etc. are contemplated as examples of possible options for inclusion in a treatment. Terms like “subject,” “user,” “patient,” or the like generally encompasses a person or entity that is provided with a treatment during the course of a trial.

One or more aspects of this disclosure pertain to a platform of a distributed messaging platform for conducting sequential randomized trials. Such trials may be provided, for example, in a scalable, efficient, and statistically rigorous manner. A platform, according to one or more aspects of this disclosure, may leverage modern computing technologies to assign treatment plans or paths to subjects, deliver treatments to subjects, collect response data, e.g., in real-time, and/or adapt to subject responses to treatments. Such operation may enable researchers to rapidly evaluate multiple interventions and determine which are most effective at promoting behavioral change, as well as efficiently select treatments for subjects that are effective on an individualized basis.

In an example use case, an entity may desire to provide a distributed messaging platform that delivers effective messages for its users. For example, various applications may include a platform for providing push notifications at various times of the day to encourage users to eat healthy snacks, to get active and interrupt a sedentary period, to take a proper medication at a prescribed time, to engage in mindfulness activities to reduce stress at various times of the day and with various messages, to encourage users to maintain proper levels of hydration, etc. A conventional trial may divide subjects into different groups, e.g., one group for each of a plurality of different messaging procedures, and then evaluate outcomes for each group at the end of the trial.

As noted above, conventional trial procedures may be sub-optimal, may not account for all factors, and/or may not be applicable to certain situations. For instance, conventional trial procedures may not be able to provide results until the end of a lengthy trial process, and moreover may lock subjects into undesirable options for the extent of the trial. Further, with conventional trial procedures, treatments are generally evaluated under the assumption that effectiveness may be understood through a mean effect, and thus may not account for individualized responses from different subjects.

To conduct a sequential randomized trial, according to one or more aspects of this disclosure, subjects of the trial may be assigned to multi-stage treatment paths. During a first portion of the trial, user devices of the subjects are caused to output a treatment corresponding to an initial first stage of their assigned treatment path. Response data is collected from the subjects, e.g., via their user devices. Subjects for whom the applied treatment was ineffective, e.g., below a predetermined threshold for effectiveness, are shifted to a next stage in their respective treatment path, such that in a next portion of the trial, the user devices of the shifted subjects are caused to instead output a treatment corresponding to the next treatment stage of their respective treatment path. Response data may be re-collected, e.g., to capture the results of the newly applied treatments. In an example, the shifting, applying of treatments, and collecting of response data is iterated, e.g., until all subjects receive an effective treatment, until all subjects who have not received an effective treatment reach an end of their respective path, for a predetermined number of iterations, etc.

This shifting through the course of the trail not only enables the application of treatments to each subject to adapt on an individual basis, but also ensures that a subject is not locked into an ineffective treatment throughout the trial. Such an approach may also improve a rate of elicited behavioral change and/or improved health outcomes. Moreover, such procedures may enable the platform to evaluate more different treatments and to reach results with statistical significance faster and/or with fewer subjects compared to conventional trial procedures.

In an example, consider a scenario where a distributed messaging platform is intended to provide emotional health improvement to participating users. In an example, a developer, to set up the trial, obtains or provides, e.g., via a user interface of a developer system, a plurality of treatments to be evaluated in the trial. In this example for providing emotional health improvement, the treatments include: (i) automated meditation and/or controlled breathing exercises output by an electronic application of a subject's user device, (ii) guided coaching, e.g., led by a licensed provider, communicated via the electronic application, (iii) a one-on-one session of cognitive behavior therapy, communicated via the electronic application, (iv) a prescription of a front-line medication, and (v) a prescription of a secondary medication. It should be understood that a treatment is not limited to a singular event or action. For example, a treatment for guided coaching encompasses scheduling guided coaching sessions once each week, providing a particular number of appointment invitations for the subject to schedule as needed, etc.

The developer, e.g., via the user interface, assigns or obtains treatment paths that incorporate the treatments above. As used herein a “treatment path” generally encompasses an ordered list that includes a plurality of treatment stages. The treatment to be applied to a particular subject is determined based on the particular treatment path assigned to that subject as well as the stage along the path that the subject is currently at. As will be discussed in further detail below, a subject may be shifted to a different treatment stage during the course of the trial, whereby the order of the ordered list defines a next treatment stage for that subject. To continue the example above, the treatment paths include:
(a) i, ii, iii, iv, v;
(b) a combination of i and ii, a combination of i, ii, iii, and iv, and a combination of i, ii, iii, and v; and
(c) iv, a combination of iii and iv, and a combination of iii and v.

In a further example, one or more treatment paths are determined automatically, e.g., based on characteristics of the treatments for the trial. For instance, one path may order treatments based on level of intervention, based on cost, based on historical treatment data, etc.

In an example, a sample size for the trial is determined, e.g., based on one or more aspects of the treatments and/or treatment paths such as highest number of treatments in a path, predetermined engagement or effectiveness data for the treatments, etc. As would be understood by one of ordinary skill in the art, sequential operations, such as the successive treatments along a treatment path, risk introduction of truncation bias and/or sample attenuation. For instance, only subjects that were shifted across all prior stages will reach a last stage in a treatment path, and thus the available sample size for treatments may diminish farther along a treatment path they are placed. One technique to address this is to ensure that a sufficient number of samples are included at the beginning of each path such that it is likely that a statistically sufficient number of samples will reach the end of the path. Other techniques are discussed in further detail below. A quantity of subjects meeting the determined sample size is obtained, e.g., by identifying user devices associated with a sufficient quantity of subjects.

The developer, in an example, also sets a metric for evaluating the effectiveness of treatments for the trial. In this example, this metric includes an evaluation of the subject using the Hamilton Depression Rating Scale (HDRS). In another example, the developer sets a desired level of statistical significance for the trial. In a further example, the developer sets an iteration interval for the trial, as discussed in further detail below.

Each subject in the trial is associated with or assigned to a respective treatment path, and is initialized at a first stage of the respective treatment path. The user device of each subject is caused to output a respective treatment corresponding to the first treatment stage of their respective treatment path. For example, a subject A is assigned to path (b), and thus a user device of subject A is caused to initiate the treatment(s) in the first stage of that path: treatment (i) automated meditation, as well as treatment (ii) a guided coaching session.

Throughout the trial, the platform may collect response data from the subject's user devices. For example, the user devices may provide data to the platform, e.g., whether a subject viewed a transmitted message, whether the subject initiates or completes a communication session via the electronic application, etc. The platform may also record other data, such as whether the subject's device was available for receiving messages when messages were sent, etc., that may be collected with or without intervention or interaction from the user device. In an example, response data may be streamed, e.g., to a database or data lake, such that results of the trial may be collected, accessed, and/or analyzed in rear or near-real time (hereafter referred to as “real-time”). Such results data may be used to determine an effectiveness of one or more of the different treatments.

At a predetermined period of time, e.g., after the iteration interval, the platform identifies whether the treatments applied to each subject were effective, e.g., by causing each subject to be evaluated via the HDRS. Subjects that received ineffective treatments are identified, and are shifted to a next stage of their respective treatment paths. For instance, Subject A is shifted to the next stage of path (b).

The subject's user devices (e.g., both those that were shifted and those that were not) are again caused to output their respective treatments. For instance, the user device of Subject A, in addition to maintaining (i) the automated meditation, as well as (ii) the guided coaching session, is caused to initiate (iii) a one-on-one session of cognitive behavior therapy, and is caused to enter and/or request (iv) a prescription of a front-line medication. As noted above, results data is continued to be collected throughout the trial.

At the end of another iteration interval, the effectiveness of treatments on the subjects is re-evaluated, and the shifting of subjects that received ineffective treatments is repeated. For instance, if the combination of (i), (ii), (iii), and (iv) was effective for Subject A, Subject A may not be shifted. But, if the combination was ineffective, Subject A is shifted to the next stage in their path for the next iteration period. Iterations continue until the trial concludes, e.g., until a predetermined number of iterations are complete, after a predetermined period of time, etc.

In some instances, subjects that received an effective treatment are not shifted to a different treatment stage for a next iteration period. In some instances, subjects that received an effective treatment are shifted to a different treatment path, e.g., to introduce micro-randomization into the trial. In some instances, the shifting of subjects that received an effective treatment is performed after a plurality of iteration intervals, e.g., so that long term data on the effective treatment is collected.

In some instances, all of the treatments along a path may be ineffective for a particular subject. In some instances, the next treatment stage for such a subject may be defined on an individualized basis, such as a most effective of the prior applied treatments. In some instances, the next treatment stage is defined as the first stage in a different treatment path. In some instances, the next treatment stage is defined as the current treatment stage, such that the subject is no longer shifted. In some instances, the subject is removed from the trial.

In some instances, the shifting of a subject to a different path, as in the examples below, is performed randomly. In some cases, the cross-path shifting is configured to gradually bias subjects toward paths and/or treatments that exhibit more effective results, such that, over the course of the trial, more and more subjects are routed into higher-performing treatments. One example of such a redistribution procedure is Thompson bandit sampling.

Results for the trial may include, for example, indications of one or more of near-term effectiveness of treatments, long-term effectiveness of treatments, response trends over time, the effect of one or more time-varying factors on engagement rate, or other factors associated with subject responses. For example, the platform may collect data regarding messaging burnout, e.g., data indicative that a subject stopped engaging with the app, e.g., based on a lack of activity, an uninstallation of the app, a change in notification settings, etc., which may be correlated against different treatments or aspects thereof such as frequency of messaging, etc. Results may be output on a user device associated with the developer, stored in a database, analyzed, e.g., using one or more automatic algorithms, etc.

While several of the examples above involve different messaging apps, it should be understood that techniques according to this disclosure are adaptable to any suitable type of distributed messaging platform. Moreover, the techniques disclosed herein are adaptable to any suitable communications platform. In an example, instead of messages, aspects of a sequential randomized trial may be applied to any suitable treatment, e.g., prescription of medication, service of media or content, advertising, etc. Further, effectiveness may be evaluated with reference to any suitable criteria, e.g., financial transactions or status, actions or activities, location, timing of an event, program or application use, etc. It should also be understood that the examples above are illustrative only.

Presented below are various aspects of implementation of a sequential randomized trial, according to various embodiments.

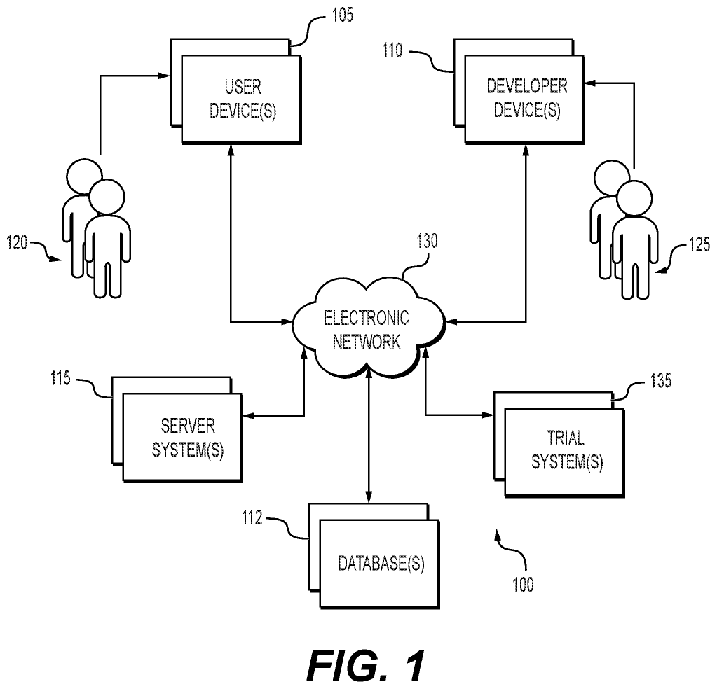

FIG. 1 depicts an example environment 100 that is utilized with sequential randomized trial techniques presented herein. One or more user device(s) 105, one or more developer device(s) 110, and one or more server system(s) 115 are configured to communicate across an electronic network 130. As will be discussed in further detail below, one or more trial system(s) 135 are configured to communicate with one or more of the other components of the environment 100 across the electronic network 130. The one or more user device(s) 105 is associated with a subject 120, e.g., a person or entity identified for participation in a sequential randomized trial. The one or more developer device(s) 110 is associated with a developer 125, e.g., a person or entity involved in providing the sequential randomized trial.

In some embodiments, one or more of the components of the environment 100 are associated with a common entity, e.g., an insurance provider, a medical care provider such as a hospital, a commercial entity, and advertiser, or the like. In some embodiments, one or more of the components of the environment 100 is associated with a different entity than another. The systems and devices of the environment 100 are configured to communicate in any arrangement. As will be discussed herein, systems and/or devices of the environment 100 are configured to communicate in order to one or more of provide goods or services, e.g., in the form of treatments, to subjects 120, provide a sequential randomized trial, or collect, store and/or analyze data associated with such a trial, among other activities.

The user device 105 is configured to enable the user 120 to access and/or interact with other systems in the environment 100. For example, the user device 105 is a computer system such as, for example, a desktop computer, a mobile device, a tablet, a wearable device such as a fitness tracker, smart watch, etc. In some embodiments, the user device 105 includes one or more electronic application(s), e.g., a program, browser, etc., installed on a memory of the user device 105. In some embodiments, the electronic application(s) is associated with or enable the user 120 to interact with one or more of the other components in the environment 100. For example, the electronic application(s) includes a browser or application configured to receive and output treatments, e.g., messages and/or other media, provided by the server system 115. For example, the user device 105, in some embodiments, includes a User Interface (UI) configured to receive and display push notifications, e.g., an alert such as a pop-up or other message that is generated even when a corresponding program or application is not open or in focus. In some embodiments, a first user device 105, e.g., a fitness tracker, is configured to interact with a second user device 105, e.g., a smartphone. In an example, a smartphone includes an electronic application with a user interface that enables interaction between a user 120 and a program or application operating on a fitness tracker. As used herein, any such combination of devices is considered a user device. In another example, the electronic application is configured to initiate a communication with another person, e.g., a text, voice, and/or video conversation.

The developer device 110 includes, for example, a user interface that enables the developer 125 to interact with one or more of the components in the environment 100. In an example, the user interface includes one or more interface elements for generating, monitoring, or analyzing a sequential randomized trial or data associated therewith. In an example, the developer device 110 includes a tool or algorithm for one or more operations such as, for example, setting or determining a statistical significance of data, determining a sample size, identifying subjects to be used for a trial, setting parameters for a trial such as trial name, trial start date, number of cohorts, treatments options, treatment paths, assignment of treatment paths to subjects, an iteration interval for the trial and/or determination of dates, frequency, or periods for checking results and/or evaluating statistical significance, etc. In some embodiments, such information is stored in a database or the like, e.g., in a memory of the trial system 135. In some embodiments, the developer device 110 interacts with or is at least partially integrated into the trial system 135. In some embodiments, the developer device 110 includes and/or interacts with an application programming interface (API), e.g., for exchanging data to other systems, e.g., one or more of the other components of the environment such as the trial system 135.

The server system 115, in various embodiments, hosts, implements, tracks, and/or facilitates services or procedures relating to a good or service to be provided to users 120. In an example, to continue from one of the examples above, the server system 115 hosts a distributed messaging service configured to provide emotional health improvement to users 120. In other embodiments, the server system 115 is configured to generate or output any other type of treatment, e.g., medical prescription information, advertising content, media, etc. The server system 115, in some embodiments, stores instructions for delivering such treatments, and/or user data such as subscription information, profile information, preference information, payment information, etc. The server system 115, in some cases, is configured to interact, e.g., via an API or the like, with the user device 105 in order to provide such treatments, e.g., via a push notification or the like. The server system 115, in some cases, is configured to interact, e.g., via an API or the like, with the developer device 110 in order to obtain a protocol for a treatment, subject data for directing a treatment, or the like, or to provide response data, usage data for the good or service hosted by the server system 115, or the like.

For instance, a user 120 may subscribe for a messaging service, e.g., via a user device 105. The user 120, in some instances, uses an electronic application operating on the user device 105 to setup a profile or account that is hosted by or in communication with the server system 115. The server system 115 determines a messaging protocol for the user 120. In an example, the server system 115 determines whether the user 120 has been selected for participation in a trial, e.g., via an API interaction with the trial system 135 or the developer device 110 or the like, and setup and implement a messaging protocol as appropriate. The server system 115 then implements the messaging protocol to provide messages to the user device 105 as directed. As discussed in further detail below, in various embodiments, the server system 115 interacts with or receives instructions from other components of the environment 100 that modify how the server system 115 operates, e.g., how messaging is provided to the user 120.

In various embodiments, the electronic network 130 is a wide area network (“WAN”), a local area network (“LAN”), personal area network (“PAN”), or the like. In some embodiments, electronic network 130 includes the Internet, and information and data provided between various systems occurs online. “Online” means connecting to or accessing source data or information from a location remote from other devices or networks coupled to the Internet. Alternatively, “online” refers to connecting or accessing an electronic network (wired or wireless) via a mobile communications network or device. The Internet is a worldwide system of computer networks-a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that includes data configured to cause a program such as a web browser to perform operations such as send, receive, or process data, generate a visual display and/or an interactive interface, or the like.

As discussed in further detail below, the trial system 135, in embodiments, includes various modules, algorithms, or the like for performing various operations. While certain operations are described in association with one or more particular modules or algorithms, it should be understood that such operations may be distributed across any suitable number of algorithms or modules, and/or multiple operations may be performed by a single module or algorithm. In an example, the trial system 135 includes one or more modules or algorithms for setting up a sequential randomized trial, for running the trial, for monitoring the trial, for analyzing response data for the trial, for generating results data for the trial, or analyzing the results data for the trial. Setting up the trial may be based on, for example, information obtained from or via the developer device 110 or the like and, in examples, includes initializing a database for the trial, determining a sample size for the trial, generating or obtaining instructions for application of one or more treatments, assigning treatments to treatment paths, populating a trial database, e.g., with subject data, assigning subjects to treatment paths, etc. Running the trial includes, for example, providing the server system 115 with appropriate treatment instructions for the subjects and/or causing different treatments to be applied to different subjects, identifying whether each subject received an effective treatment, shifting subjects that received ineffective treatments to a next treatment stage, e.g., at an end of each iteration interval of the trial, collecting response data from the subjects, etc. Generating and/or analyzing results includes, for example, sorting, modeling, and/or applying one or more statistical procedure to response data. Example procedures include evaluating trial results using a linear mixed effects model, or the like.

In some embodiments, the trial system 135 includes or has access to a database or data lake 112 configured to act as a repository or source for data such as, data associated with a trial, data obtained via the user interface of the developer device 110, or data obtained from any other suitable source, e.g., treatment data or protocol information, treatment path data, trial subject information, response data, subject engagement data, subject demographic data, etc. In some embodiments, such data may be stored and/or accessed by one or more other components of the environment 100, such as the developer device 110, the server system 115, or the like.

In some embodiments, treatment data or protocol information includes, for example, contents of a treatment, e.g., a message or series of messages, a prescription or course, a directed interaction or series of interactions or appointments, delivery of a good, etc. In some instances, a treatment has multiple steps, e.g., a series of sequential messages, or includes a combination of options, such as a workout reminder message every day and a web meeting with a trainer once a week.

In some embodiments, treatment data or protocol information includes criteria or conditions for providing one or more aspects of the treatment. In an example, provision of a prescription of a medication is associated with a criterion that a subject is not allergic to that medication (e.g., based on subject medical history data). In another example, scheduling of a communication such as a group therapy or one-on-one counselling session is associated with a criterion relating to the subject's calendar or preferences. Other example criteria in various embodiments includes, for example, a subject's location (e.g., as indicated by positioning data of their user device), message or treatment frequency, indications of user activity, historical actions taken by the subject 120, an indication of notification fatigue for the subject 120, etc. In an example, a protocol includes an instruction that a reminder message not be sent if the subject has takin a particular action recently or with a threshold amount of frequency. In another example, a protocol includes an instruction not to send a reminder message in response to determining that the subject 120 is traveling away from home, to weather conditions not being amiable to the recommended action, to determining that the subject 120 has cancelled an associated service, has a record of an illness or doctor visit, has a scheduling conflict, etc.

Although depicted as separate components in FIG. 1, it should be understood that a component or portion of a component in the environment 100 may, in some embodiments, be integrated with or incorporated into one or more other components. For example, a portion of the server system 115 is integrated into the trial system 135, or the like. In another example, a portion of the server system 115 is integrated into the user device 105, or vice versa. In some embodiments, operations or aspects of one or more of the components discussed above are distributed amongst one or more other components. Any suitable arrangement and/or integration of the various systems and devices of the environment 100 is usable in various embodiments. In one embodiment, one or more components of the environment are implemented as a cloud service or at least partially on a cloud environment, e.g., the server system 115, the trial system 135, etc.

In the methods below, various acts are described as performed or executed by a component from the environment 100 of FIG. 1, such as the trial system 135, the server system 115, the developer device 110, the user device 105, or components thereof. However, it should be understood that in various embodiments, various components of the computing environment 100 discussed above may execute instructions or perform acts including the acts discussed below. Moreover, an operation described as implemented by a particular component may also be understood to refer to execution of that operation by a corresponding module or algorithm, e.g., by one or more processors associated with that component or others. Further, it should be understood that in various embodiments, various steps may be added, omitted, and/or rearranged in any suitable manner.

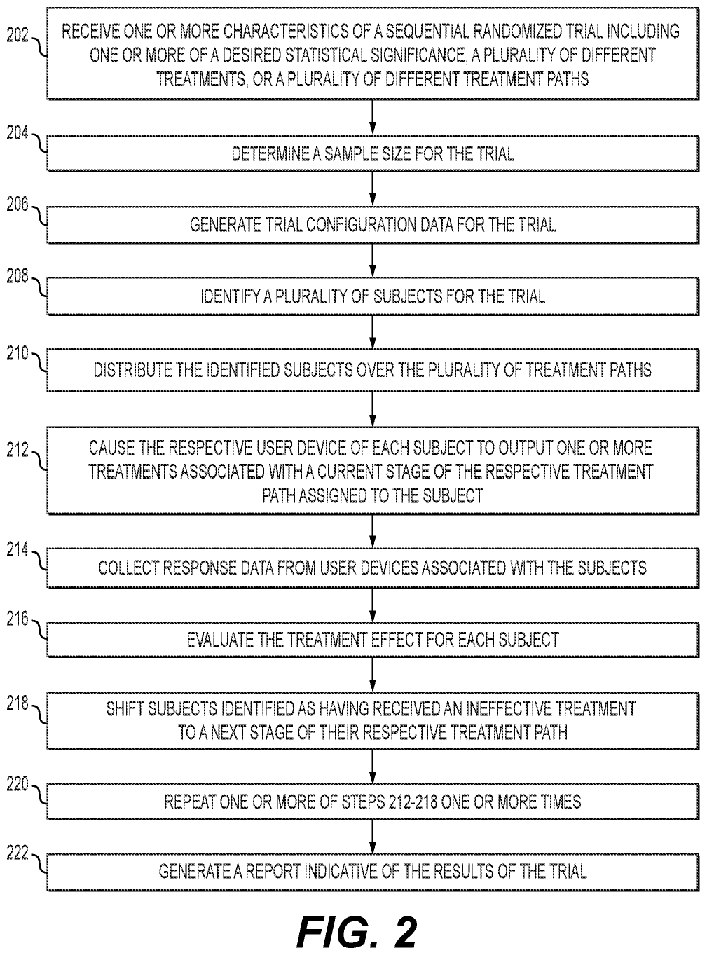

FIG. 2 illustrates an example process for implementing a sequential randomized trial, according to one or more aspects of this disclosure. A developer 125 or an associated entity may desire to understand the effectiveness for different options in a provided good or service. In some instances, the good or service may already be provided, and thus a comparison is desired between an existing practice, e.g., a control, and a plurality of different options. In some instances the good or service is not yet available, and thus a comparison is desired between a plurality of different options. The comparison includes implementation of a sequential randomized trial. In a particular example, a distributed messaging platform is hosted on a server system 115, and follows a protocol to provide reminder messages to one or more user devices, e.g., that are associated with users subscribed via a reminder application. The reminder messages include causing the user devices 105 to display push notifications including the messages. In some instances, such as when a treatment includes an interaction such as a therapy session, or a provision of a prescription for a medication, the message includes an associated indication or notification, and/or a link to further information.

To implement the trial, the developer 125 accesses a user interface of a developer device 110 that is in communication with a trial system 135, e.g., via an API. At step 202, the interface receives one or more characteristics of the trial, e.g., a name, a start date/time, a plurality of experimental options to be used as treatments (with or without an option corresponding to an existing protocol as a control), a plurality of treatment paths for the treatments, assignments of one or more treatments to each treatment stage of each treatment path, as well as other information such as criteria or protocol for different treatments, criteria or protocol for how subjects are to be distributed across the different treatment paths, statistical significance or power desired for results of the trial, a period of time defining an iteration interval for the trial, a predetermined response to a subject reaching the end of a treatment path, a predetermined protocol for a subject being provided with an effective treatment, a metric for evaluating treatment effectiveness, etc. In various embodiments, the interval is daily, weekly, monthly, or any other suitable period of time. Further aspects of the redistributions are discussed below. Portions of such information, in various embodiments, are preset, user provided, determined automatically, determined randomly, or combinations thereof.

At step 204, the trial system 135 determines a sample size for the trial, e.g., based on the one or more characteristics received in step 202. The sample size, in some embodiments, is a quantity of subjects needed for results of the trial to obtain the desired statistical power or significance. In some embodiments, the sample size is determined so that a sufficient number of subjects reach each treatment stage of each treatment path. In some embodiments, the trial system 135 is further configured to determine a number of intervals needed for running the trial. In an example, embodiment, the sample size is determined via any suitable procedure for determining sample sizes for studies with correlated observations, e.g., an algorithm configured to calculate upper tail probabilities for random variables with binomial distributions.

At step 206, the trial system 135 generates trial configuration data, e.g., based on the received characteristics from step 202 and/or the sample size and/or timing determined from step 204, and output the trial configuration data. In an example, the trial system 135 one or more of causes the user interface of the developer device 110 to output a display of one or more aspects of the trial configuration data, or stores the trial configuration data in the database 112, or the like.

At step 208, the trial system 135 identifies a plurality of subjects for the trial, e.g., based on the sample size determined at step 204. In some embodiments, the trial configuration data includes criteria for selection of subjects. In some embodiments, the trial system 135, in order to identify one or more user devices 105 for subjects 120 for the trial, accesses or interacts with the server system 115. In an example, the server system 115 includes profile information for subscribed users that is compared against trial subject criteria to identify subjects 120 for the trial.

At step 210, the trial system 135 distributes the identified subjects 120 over the plurality of treatment paths for the trial. In an example, the trial system 135 determines a respective probability for each subject 120, and then determine a respective cohort for the subject 120 based on the determined respective probability. In some embodiments, the respective probability accounts for criteria associated with the treatment paths. In some embodiments, the distribution is random or pseudo-random, e.g., based on the output of a pseudo-random number generator.

At step 212, the trial system 135 causes the respective user device 105 of each subject 120 to output one or more treatments associated with a current stage of the respective treatment path assigned to the subject 120. In one example, the trial system 135 identifies a treatment procedure or protocol for each subject 120 to the server system 115, and the server system 115 implements such procedure or protocol. As noted above, a treatment may have multiple steps or branches, which may have one or more criteria. In other words, each treatment may range from a singular act to a campaign with multiple decision points. In some embodiments, treatments to subjects 120 are provided in real time and/or streamed to the user device 105.

At step 214, the trial system 135 collects response data from user devices 105 associated with the subjects 120. In some embodiments, collected response data is aggregated by trial iteration interval. In some embodiments, collected response data is received in real time, may be streamed to the database 112, or the like. In some embodiments, the trial system 135 is configured to request response data from the user devices 105. In some embodiments, an electronic application or program operating on the user devices 105 is configured to periodically provide response data to the trial system 135 and/or are configured to provide response data in response to an interaction with the subject 120. In an example, the user device 105 is configured to automatically provide response data in response to the subject 120 one or more of engaging with a message, taking or reporting completion of a particular action, being in a particular location, etc. Response data, in an example, includes one or more of an identification of the subject, an identification of the treatment path, treatment stage, or of the treatment provided, an index value indicative of whether or when a particular treatment was provided, data indicative of engagement of the subject 120 with the treatment, an availability of the subject 120 and/or the user device 105 when the treatment was provided (e.g., was the user device 105 on and in service), an indication of whether the subject 120 received the treatment, data associated with reporting or an indication of a response or outcome from the treatment, etc.

At step 216, e.g., at an end of an iteration interval, the trial system 135 evaluates the treatment effect on each subject 120. In an example, the trial system 135 evaluates the response data for each subject 120 with reference to the evaluation metric for the trial. In an example, the trial system 135 identifies which subjects 120 received an effective treatment, e.g., a treatment resulting in an evaluation metric above a predetermined threshold, and which subjects 120 received an ineffective treatment, e.g., a treatment resulting in an evaluation metric below the predetermined threshold. Such evaluation data is, for example, streamed and/or stored in the database 112.

At step 218, the trial system 135 shifts the subjects 120 identified as having received an ineffective treatment to a next stage in their respective treatment paths.

At step 220, one or more of steps 212-218 are repeated one or more times. In other words, the causing of the application of treatments, the collecting and evaluating of response data, and the shifting of any subjects 120 that received an ineffective treatment is repeated throughout the trial, e.g., over each successive iteration interval of the trial. It should be understood, however, that in some embodiments, one or more of the steps 214-218 are performed concurrently. In an example, collection of response data continues throughout a trial, e.g., rather than being conducted as a discrete step at a discrete moment in time.

Moreover, in some instances, a subject 120 is shifted to a next stage prior to an end of the iteration interval. For example, if a subject has a very negative response to a treatment early on in an iteration interval, the subject 120 can be shifted to a next stage early. In another example, the user device 105 of the subject is configured to receive a request from the subject 120 to receive a different treatment or reject a current treatment, etc.

In some instances, subjects 120 that received an effective treatment are not shifted to a different treatment stage for a next iteration period. In some instances, subjects 120 that received an effective treatment are shifted to a different treatment path, e.g., to introduce micro-randomization into the trial. In some instances, the shifting of subjects 120 that received an effective treatment is performed after a plurality of iteration intervals, e.g., so that long term data on the effective treatment is collected.

In some instances, all of the treatments along a path may be ineffective for a particular subject 120. In some instances, the next treatment stage for such a subject 120 is defined on an individualized basis, such as a most effective of the prior applied treatments. In some instances, the next treatment stage is defined as the first stage in a different treatment path. In some instances, the next treatment stage is defined as the current treatment stage, such that the subject is no longer shifted. In some instances, the subject 120 is removed from the trial.

In some instances, the shifting of a subject 20 to a different path is performed randomly. In some cases, the cross-path shifting is configured to gradually bias subjects 120 toward paths and/or treatments that exhibit more effective results, such that, over the course of the trial, more and more subjects 120 are routed into higher-performing treatments. One example of such a redistribution procedure is Thompson bandit sampling.

The iteration of the one or more of the steps 212-218 is repeated until the trial reaches an end point. In one example, the end point occurs when all subjects 120 have received an effective treatment. In another example, the end point occurs when all subjects that have received ineffective treatments reach the end of their respective treatment paths. In a further example, the end point occurs after a predetermined number of iteration intervals. Any other suitable end point may be used.

At step 222, the trial system 135 generates a report indicative of the results of the trial. Such a report, in various embodiments, includes content such as, for example, an impact or near-term effectiveness of different treatments, an impact of characteristics of the subjects 120 and/or time-varying factors on the effectiveness of the different treatments, long term effectiveness of the different treatments, etc. In various embodiments, such content is generated automatically and/or via human analysis, e.g., by performing statistical examination of the results. In one aspect, effectiveness for a treatment is determined at various granularities, e.g., just after application, within an hour, five hours, a day, a week, over time, etc. In another aspect, trends may be identified. In an example, correlations between long term behavior changes in the subjects 120 and a sequence of different treatments are identified. In an example, the trial system 135 collects data regarding messaging burnout, e.g., data indicative that a subject stopped engaging with the app, e.g., based on a lack of activity, an uninstallation of the app, a change in notification settings, etc., which may be correlated against different treatments or aspects thereof such as frequency of messaging, etc.

In an example, the trail system 135 analyzes the results of the trial using a linear mixed effects model, e.g., with random intercept and fixed slopes. One of the advantages of using such a model is that all model parameters are estimated in the presence of missing data, removing the requirement for data estimation and/or case-wise deletion. The results and/or analysis is, for example, stored in the database 112. In another example, results and/or analysis are used to generate a report that is output to the developer 125, e.g., via the user interface of the developer system 110.

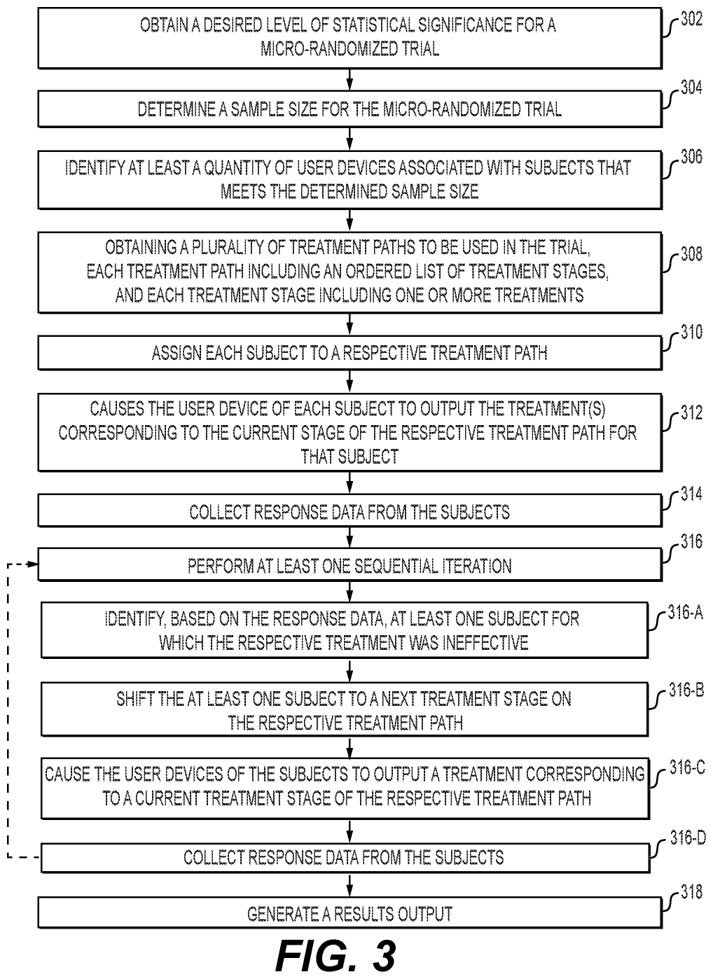

FIG. 3 depicts another example embodiment of a process for implementing a sequential randomized trial, according to one or more aspects of this disclosure. A developer 125 or an associated entity may desire to conduct a sequential randomized trial, e.g., in order to evaluate different options for providing a good or service. At step 302, a trial system 135 obtains, e.g., via a user interface of a developer device 110 or a preset value or the like, a desired level of statistical significance for the trial. At step 304, the trial system 135 determines a sample size for the sequential randomized trial, e.g., based on the desired level of statistical significance and/or other factors.

At step 306, the trial system 135 identifies, e.g., via interaction with a server system 115 over an electronic network, at least a quantity of user devices 105 associated with subjects 120 that meets the determined sample size. In an example, subjects 120 are identified according to one or more criteria for the trial, e.g., subscribed to be provided with the good or service associated with the trial, agreed to opt in to the trial, available for the trial, in a geographical region approved for the trial, over a threshold minimum age, etc. The trial system 135, in an embodiment, additionally sets or obtains one or more characteristics for the trial such as, for example, a start day/time, a period of time to be used as in iteration interval for the trial, a number of iteration intervals, an end point criterion, a treatment effectiveness metric, etc.

At step 308, the trial system 135 obtains a plurality of treatment paths to be used in the sequential randomized trial. In an example, each treatment path includes an ordered list of a plurality of treatment stages, and each treatment stage includes one or more treatments. The treatments, treatment stages, and treatment paths are, for example, set or obtained by the developer 125 via the user interface of the developer device 110.

At step 310, the trial system 135 assigns each of the plurality of subjects 120 into a respective treatment path. In an example, each subject 120 is initialized at a first stage of their respective treatment path. In some embodiments, the assignment is performed randomly. In some embodiments, the likelihood of being assigned to each treatment path is the same as any other. In some embodiments, criteria of the treatment paths and/or characteristics of the subjects 120 bias the subjects toward or away from one or more treatment paths.

At step 312, the trial system 135 causes the user device 105 of each subject 120 to output the treatment(s) corresponding to the current stage of the respective treatment path for that subject 120. In some embodiments, the trial system 135 provides, for example, instructions to the server system 115 for applying different treatments to different subjects 120, whereby the server system 115 causes user devices 105 of the subjects 120 to generate, output, direct, and/or apply the corresponding treatments to the subjects 120. It should be understood, however, that this arrangement is an example only, and that any suitable arrangement may be used. For example, in some embodiments, the trial system 135 communicates with the user devices 105, e.g., without a server system intermediary. In some embodiments, the trial system 135 directs another system or device to schedule delivery of a treatment at a later time. In an example, the trial system 135 causes a user device 105 to generate a push notification at various times without requiring the trial system 135 to communicate with the user device 105 at each instance.

At step 314, and e.g., throughout the trial, the trial system 135 collects, from the plurality of user devices 105, response data of the plurality of subjects 120. In some embodiments, the collected response data is indexed by time steps for the trial, e.g., by hour, day, week, etc. The time step for collecting data, in some embodiments, is an extent of time that is less than the iteration interval. In some embodiments, the response data is collected in real time, and/or is transmitted (e.g., streamed) from the user devices 105, e.g., to a database 112 or the like. The response data, in some embodiments, includes, for example, for each time step, one or more of subject engagement data, subject availability data, assigned treatment data, or near-term outcome data. The trial system 135, in an embodiment, bins or indexes response data, e.g., in a relational database, via one or more characteristic such as time step, interval, subject 120, treatment, cohort, etc.

At step 316, the trial system 135 performs at least one sequential iteration, e.g., over a subsequent iteration interval. In an example, an iteration includes, at step 316-A, identifying, via the trial system 135 and based on the response data, at least one subject 120 for which the respective treatment was ineffective. At step 316-B, the trial system 135 shifts the at least one subject 120 to a next treatment stage on the respective treatment path. At step 316-C, the trial system 135 causes the user devices 105 of the subjects 120 (e.g., the identified subjects 120 or all subjects 120) to output a treatment corresponding to a current treatment stage of the respective treatment path (e.g., the “next” stage that they were shifted to in step 316-B). For example, a subject that was not shifted will receive the same treatment as in step 312, whereas a subject that was shifted in step 316-B instead receives the next treatment on their treatment path. At step 13-D, the trial system 135 collects response data from the user devices associated with the subjects 120.

At step 318, the trial system 135 generates a results output. The results output includes, for example, a notification delivered to the developer device 110 indicating whether the results for at least one treatment has reached statistical significance. The results output includes, in another example, a report indicative of results of the sequential randomized trial. In another example, the report includes data indicative of an amount of impact or effectiveness of the different treatments, and/or impact on effectiveness of the treatments from one or more time-varying factors. The report, in some embodiments, includes trends, analysis, and/or conclusions that may be drawn from the results data, which in different embodiments are generated automatically via one or more algorithms and/or with the aid of a human investigator.

It should be understood that embodiments in this disclosure are examples only, and that other embodiments may include various combinations of features from other embodiments, as well as additional or fewer features. For example, while some of the embodiments above pertain to a sequential randomized trial operated in conjunction with a distributed messaging platform, any suitable sequential randomized trial with any suitable type or kind of treatment may be implemented according to one or more aspects of this disclosure.

In general, any process or operation discussed in this disclosure that is understood to be computer-implementable, such as the processes illustrated in FIGS. 2 and 3, is performed by one or more processors of a computer system, such any of the systems or devices in the environment 100 of FIG. 1, as described above. A process or process step performed by one or more processors may also be referred to as an operation. The one or more processors are configured to perform such processes by having access to instructions (e.g., software or computer-readable code) that, when executed by the one or more processors, cause the one or more processors to perform the processes. The instructions are stored in a memory of the computer system. A processor is a central processing unit (CPU), a graphics processing unit (GPU), or any suitable types of processing unit.

A computer system, such as a system or device implementing a process or operation in the examples above, includes one or more computing devices, such as one or more of the systems or devices in FIG. 1. One or more processors of a computer system are included in a single computing device or distributed among a plurality of computing devices. A memory of the computer system includes the respective memory of each computing device of the plurality of computing devices.

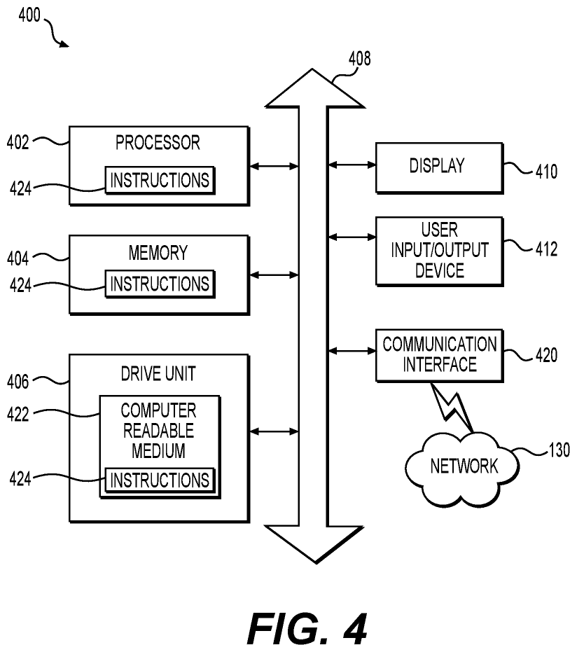

FIG. 4 is a simplified functional block diagram of a computer 400 that is configured as a device for executing the methods of FIGS. 2 and 3, according to example embodiments of the present disclosure. For example, the computer 400 is configured as the trial system 135 and/or another system according to example embodiments of this disclosure. In various embodiments, any of the systems herein is a computer 400 including, for example, a data communication interface 420 for packet data communication. The computer 400 also includes a central processing unit (“CPU”) 402, in the form of one or more processors, for executing program instructions. The computer 400 includes an internal communication bus 408, and a storage unit 406 (such as ROM, HDD, SDD, etc.) that may store data on a computer readable medium 422, although the computer 400 may receive programming and data via network communications. The computer 400 may also have a memory 404 (such as RAM) storing instructions 424 for executing techniques presented herein, although the instructions 424 are stored temporarily or permanently within other modules of computer 400 (e.g., processor 402 and/or computer readable medium 422). The computer 400 also includes input and output ports 412 and/or a display 410 to connect with input and output devices such as keyboards, mice, touchscreens, monitors, displays, etc. The various system functions are implemented in a distributed fashion on a number of similar platforms, to distribute the processing load. Alternatively, the systems is implemented by appropriate programming of one computer hardware platform.

Program aspects of the technology are thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that stores the software elements includes optical, electrical and electromagnetic waves, such as used across physical interfaces between local devices, through wired and optical landline networks and over various air-links. The physical elements that carry such waves, such as wired or wireless links, optical links, or the like, also is considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments are applicable to any environment, such as a desktop or laptop computer, an automobile entertainment system, a home entertainment system, etc. Also, the disclosed embodiments are applicable to any type of Internet protocol.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

The present disclosure furthermore relates to the following aspects.
Clause 1. A distributed messaging platform for providing a sequential randomized trial, comprising: at least one trial system that includes: at least one memory storing instructions; and at least one processor operatively connected to the at least one memory, and configured to execute the instructions to perform operations for executing the sequential randomized trial, including: obtaining a plurality of treatment paths to be used in the sequential randomized trial, each treatment path including an ordered list of a plurality of treatment stages; assigning each subject of a plurality of subjects to a respective treatment path; for each subject, causing a corresponding user device to output a respective treatment corresponding to a first treatment stage of the respective treatment path; collecting, from the user devices, response data of the plurality of subjects; and performing at least one sequential iteration, including: identifying, based on the response data, at least one subject for which the respective treatment was ineffective; shifting the at least one subject to a next treatment stage on the respective treatment path; causing the corresponding user device of the at least one subject to output a further respective treatment corresponding to a current treatment stage of the respective treatment path; and re-collecting the response data from the user device associated with the at least one subject.
Clause 2. The distributed messaging platform of clause 1, wherein the operations further include: obtaining a desired level of statistical significance for the sequential randomized trial; determining a sample size for the sequential randomized trial based on the desired level of statistical significance; and identifying, over an electronic network operatively connected to the at least one trial system, the plurality of subjects by identifying a quantity of corresponding user devices that meets the determined sample size.
Clause 3. The distributed messaging platform of any of the preceding clauses, wherein, upon a subject being shifted to an end stage of the respective treatment path, a next treatment stage of the subject is defined as: a first treatment stage of a different treatment path; the end stage of the respective treatment path; or a null treatment stage.
Clause 4. The distributed messaging platform of any of the preceding clauses, wherein collecting the response data from the plurality of user devices includes transmitting respective response data from each of the user devices in real time to a database or data lake.
Clause 5. The distributed messaging platform of any of the preceding clauses, wherein the at least one sequential iteration further includes, for at least one further subject for which the respective treatment was effective: maintaining a position of the at least one further subject at the current treatment stage of the respective treatment path; or shifting the at least one further subject to a different treatment path.
Clause 6. The distributed messaging platform of any of the preceding clauses, wherein the operations further include: analyzing results of the sequential randomized trial using a linear mixed effects model.
Clause 7. The distributed messaging platform of any of the preceding clauses, wherein the plurality of treatment paths are obtained by causing a developer device to output a user interface configured to receive treatments and assignments of treatments into treatment paths.
Clause 8. The distributed messaging platform of clause 7, wherein: the user interface is further configured to receive an identification of a primary outcome measure for the sequential randomized trial that is indicative of treatment effectiveness; and collecting the response data includes evaluating the response data against the primary outcome measure to evaluate treatment effectiveness.
Clause 9. The distributed messaging platform of any of the preceding clauses, wherein the operations further include: generating a report indicative of results of the sequential randomized trial.
Clause 10. The distributed messaging platform of any of the preceding clauses, wherein the at least one sequential iteration is performed at an end of a predetermined interval of time.
Clause 11. A computer-implemented method of using one or more processors of a distributed messaging platform to provide a sequential randomized trial, comprising: obtaining a plurality of treatment paths to be used in the sequential randomized trial, each treatment path including an ordered list of a plurality of treatment stages; assigning each subject of a plurality of subjects to a respective treatment path; for each subject, causing a corresponding user device to output a respective treatment corresponding to a first treatment stage of the respective treatment path; collecting, from the user devices, response data of the plurality of subjects; and performing at least one sequential iteration, including: identifying, based on the response data, at least one subject for which the respective treatment was ineffective; shifting the at least one subject to a next treatment stage on the respective treatment path; causing the corresponding user device of the at least one subject to output a further respective treatment corresponding to a current treatment stage of the respective treatment path; and re-collecting the response data from the user device associated with the at least one subject.
Clause 12. The computer-implemented method of clause 11, further comprising: obtaining a desired level of statistical significance for the sequential randomized trial; determining a sample size for the sequential randomized trial based on the desired level of statistical significance; and identifying, over an electronic network, the plurality of subjects by identifying a quantity of corresponding user devices that meets the determined sample size.
Clause 13. The computer-implemented method of any of clauses 11-12, wherein, upon a subject being shifted to an end stage of the respective treatment path, a next treatment stage of the subject is defined as: a first treatment stage of a different treatment path; the end stage of the respective treatment path; or a null treatment stage.
Clause 14. The computer-implemented method of any of clauses 11-13, wherein collecting the response data from the plurality of user devices includes transmitting respective response data from each of the user devices in real time to a database or data lake.
Clause 15. The computer-implemented method of any of clauses 11-14, wherein the at least one sequential iteration further includes, for at least one further subject for which the respective treatment was effective: maintaining a position of the at least one further subject at the current treatment stage of the respective treatment path; or shifting the at least one further subject to a different treatment path.
Clause 16. The computer-implemented method of any of clauses 11-15, further comprising: analyzing results of the sequential randomized trial using a linear mixed effects model.
Clause 17. The computer-implemented method of any of clauses 11-16, wherein the plurality of treatment paths are obtained by causing a developer device to output a user interface configured to receive treatments and assignments of treatments into treatment paths.
Clause 18. The computer-implemented method of any of clauses 11-17, wherein: the user interface is further configured to receive an identification of a primary outcome measure for the sequential randomized trial that is indicative of treatment effectiveness; and collecting the response data includes evaluating the response data against the primary outcome measure to evaluate treatment effectiveness.
Clause 19. The computer-implemented method of any of clauses 11-18, further comprising: generating a report indicative of results of the sequential randomized trial.
Clause 20. A non-transitory computer-readable medium comprising instructions for using a distributed messaging platform to provide a sequential randomized trial, the instructions executable by at least one processor to perform operations including: obtaining a plurality of treatment paths to be used in the sequential randomized trial, each treatment path including an ordered list of a plurality of treatment stages; assigning each subject of a plurality of subjects to a respective treatment path; for each subject, causing a corresponding user device to output a respective treatment corresponding to a first treatment stage of the respective treatment path; collecting, from the user devices, response data of the plurality of subjects; and performing at least one sequential iteration, including: identifying, based on the response data, at least one subject for which the respective treatment was ineffective; shifting the at least one subject to a next treatment stage on the respective treatment path; causing the corresponding user device of the at least one subject to output a further respective treatment corresponding to a current treatment stage of the respective treatment path; and re-collecting the response data from the user device associated with the at least one subject.

What is claimed is:

1. A distributed messaging platform for providing a sequential randomized trial, comprising:
at least one trial system that includes:
at least one memory storing instructions; and
at least one processor operatively connected to the at least one memory, and configured to execute the instructions to perform operations for executing the sequential randomized trial, including:
obtaining a plurality of treatment paths to be used in the sequential randomized trial, each treatment path including an ordered list of a plurality of treatment stages;
assigning each subject of a plurality of subjects to a respective treatment path;
for each subject, causing a corresponding user device to output a respective treatment corresponding to a first treatment stage of the respective treatment path;
collecting, from the user devices, response data of the plurality of subjects; and
performing at least one sequential iteration, including:
identifying, based on the response data, at least one subject for which the respective treatment was ineffective;
shifting the at least one subject to a next treatment stage on the respective treatment path;
causing the corresponding user device of the at least one subject to output a further respective treatment corresponding to a current treatment stage of the respective treatment path; and
re-collecting the response data from the user device associated with the at least one subject.

2. The distributed messaging platform of claim 1, wherein the operations further include:
obtaining a desired level of statistical significance for the sequential randomized trial;
determining a sample size for the sequential randomized trial based on the desired level of statistical significance; and
identifying, over an electronic network operatively connected to the at least one trial system, the plurality of subjects by identifying a quantity of corresponding user devices that meets the determined sample size.

3. The distributed messaging platform of claim 1, wherein, upon a subject being shifted to an end stage of the respective treatment path, a next treatment stage of the subject is defined as:
a first treatment stage of a different treatment path;
the end stage of the respective treatment path; or
a null treatment stage.

4. The distributed messaging platform of claim 1, wherein collecting the response data from the plurality of user devices includes transmitting respective response data from each of the user devices in real time to a database or data lake.

5. The distributed messaging platform of claim 1, wherein the at least one sequential iteration further includes, for at least one further subject for which the respective treatment was effective:
maintaining a position of the at least one further subject at the current treatment stage of the respective treatment path; or
shifting the at least one further subject to a different treatment path.

6. The distributed messaging platform of claim 1, wherein the operations further include:
analyzing results of the sequential randomized trial using a linear mixed effects model.

7. The distributed messaging platform of claim 1, wherein the plurality of treatment paths are obtained by causing a developer device to output a user interface configured to receive treatments and assignments of treatments into treatment paths.

8. The distributed messaging platform of claim 7, wherein:
the user interface is further configured to receive an identification of a primary outcome measure for the sequential randomized trial that is indicative of treatment effectiveness; and
collecting the response data includes evaluating the response data against the primary outcome measure to evaluate treatment effectiveness.

9. The distributed messaging platform of claim 1, wherein the operations further include:
generating a report indicative of results of the sequential randomized trial.

10. The distributed messaging platform of claim 1, wherein the at least one sequential iteration is performed at an end of a predetermined interval of time.

11. A computer-implemented method of using one or more processors of a distributed messaging platform to provide a sequential randomized trial, comprising:
obtaining a plurality of treatment paths to be used in the sequential randomized trial, each treatment path including an ordered list of a plurality of treatment stages;
assigning each subject of a plurality of subjects to a respective treatment path;
for each subject, causing a corresponding user device to output a respective treatment corresponding to a first treatment stage of the respective treatment path;
collecting, from the user devices, response data of the plurality of subjects; and
performing at least one sequential iteration, including:
identifying, based on the response data, at least one subject for which the respective treatment was ineffective;
shifting the at least one subject to a next treatment stage on the respective treatment path;
causing the corresponding user device of the at least one subject to output a further respective treatment corresponding to a current treatment stage of the respective treatment path; and
re-collecting the response data from the user device associated with the at least one subject.

12. The computer-implemented method of claim 11, further comprising:
obtaining a desired level of statistical significance for the sequential randomized trial;
determining a sample size for the sequential randomized trial based on the desired level of statistical significance; and
identifying, over an electronic network, the plurality of subjects by identifying a quantity of corresponding user devices that meets the determined sample size.

13. The computer-implemented method of claim 11, wherein, upon a subject being shifted to an end stage of the respective treatment path, a next treatment stage of the subject is defined as:
a first treatment stage of a different treatment path;
the end stage of the respective treatment path; or
a null treatment stage.

14. The computer-implemented method of claim 11, wherein collecting the response data from the plurality of user devices includes transmitting respective response data from each of the user devices in real time to a database or data lake.

15. The computer-implemented method of claim 11, wherein the at least one sequential iteration further includes, for at least one further subject for which the respective treatment was effective:
maintaining a position of the at least one further subject at the current treatment stage of the respective treatment path; or
shifting the at least one further subject to a different treatment path.

16. The computer-implemented method of claim 11, further comprising:
analyzing results of the sequential randomized trial using a linear mixed effects model.

17. The computer-implemented method of claim 11, wherein the plurality of treatment paths are obtained by causing a developer device to output a user interface configured to receive treatments and assignments of treatments into treatment paths.

18. The computer-implemented method of claim 17, wherein:
the user interface is further configured to receive an identification of a primary outcome measure for the sequential randomized trial that is indicative of treatment effectiveness; and
collecting the response data includes evaluating the response data against the primary outcome measure to evaluate treatment effectiveness.

19. The computer-implemented method of claim 11, further comprising:
generating a report indicative of results of the sequential randomized trial.

20. A non-transitory computer-readable medium comprising instructions for using a distributed messaging platform to provide a sequential randomized trial, the instructions executable by at least one processor to perform operations including:
obtaining a plurality of treatment paths to be used in the sequential randomized trial, each treatment path including an ordered list of a plurality of treatment stages;
assigning each subject of a plurality of subjects to a respective treatment path;
for each subject, causing a corresponding user device to output a respective treatment corresponding to a first treatment stage of the respective treatment path;
collecting, from the user devices, response data of the plurality of subjects; and
performing at least one sequential iteration, including:
identifying, based on the response data, at least one subject for which the respective treatment was ineffective;
shifting the at least one subject to a next treatment stage on the respective treatment path;
causing the corresponding user device of the at least one subject to output a further respective treatment corresponding to a current treatment stage of the respective treatment path; and
re-collecting the response data from the user device associated with the at least one subject.
